(19) United States
(12) Patent Application Publication
(10) Pub. No.: US 2026/0021349 A1
(43) Pub. Date: Jan. 22, 2026

(54) TRAINING ASSESSMENT METHOD, ELECTRONIC DEVICE, AND COMPUTER-READABLE STORAGE MEDIUM

(71) Applicant: GUANGDONG COROS SPORTS TECHNOLOGY CO., LTD, Dongguan (CN)

(72) Inventors: Shuhui MA, Dongguan (CN); Xin LIU, Dongguan (CN)

(21) Appl. No.: 18/778,063

(22) Filed: Jul. 19, 2024

(51) Int. Cl. A63B 24/00; G16H 20/30

(52) U.S. Cl. CPC A63B 24/0075; G16H 20/30; A63B 2220/22; A63B 2220/62

(57) ABSTRACT

A training assessment method includes obtaining motion data of a user when executing a preset training plan, and determining an achievement rate and a completion rate corresponding to the user based on the motion data and the preset training plan. The achievement rate corresponding to the user characterizes an achievement situation of the user in terms of training intensity, and the completion rate corresponding to the user characterizes a completion situation of the user in terms of training volume. The method further includes determining an assessment result based on the achievement rate corresponding to the user and the completion rate corresponding to the user.

### TECHNICAL FIELD

The present disclosure generally relates to the field of terminal technologies and, more particularly, to a training assessment method, an electronic device, and a computer-readable storage medium.

### BACKGROUND

During fitness training, it is needed to understand training status of a user, to assess the rationality of a training plan based on the training status, such that the training plan can be adjusted in a timely and effective manner to improve the training effect. A current training assessment method generally includes obtaining the amount of the training plan executed by the user (such as time and/or distance, etc.) to assess the user's training status based on the user's training amount. That is, the current training assessment method only performs assessment based on the training amount, and the assessment information is relatively unilateral and cannot fully reflect the user's training status, resulting in poor accuracy and effect of the training assessment.

### SUMMARY

In accordance with the present disclosure, there is provided a training assessment method including obtaining motion data of a user when executing a preset training plan, and determining an achievement rate and a completion rate corresponding to the user based on the motion data and the preset training plan. The achievement rate corresponding to the user characterizes an achievement situation of the user in terms of training intensity, and the completion rate corresponding to the user characterizes a completion situation of the user in terms of training volume. The method further includes determining an assessment result based on the achievement rate corresponding to the user and the completion rate corresponding to the user.

Also in accordance with the present disclosure, there is provided an electronic device including at least one memory storing at least one computer program, and at least one processor configured to execute the at least one computer program to obtain motion data of a user when executing a preset training plan, and determine an achievement rate and a completion rate corresponding to the user based on the motion data and the preset training plan. The achievement rate corresponding to the user characterizes an achievement situation of the user in terms of training intensity, and the completion rate corresponding to the user characterizes a completion situation of the user in terms of training volume. The at least one processor is further configured to execute the at least one computer program to determine an assessment result based on the achievement rate corresponding to the user and the completion rate corresponding to the user.

Also in accordance with the present disclosure, there is provided a non-transitory computer readable storage medium storing at least one computer program that, when executed by at least one processor, causes the at least one processor to obtain motion data of a user when executing a preset training plan, and determine an achievement rate and a completion rate corresponding to the user based on the motion data and the preset training plan. The achievement rate corresponding to the user characterizes an achievement situation of the user in terms of training intensity, and the completion rate corresponding to the user characterizes a completion situation of the user in terms of training volume. The at least one computer program that, when executed by at least one processor, further causes the at least one processor to determine an assessment result based on the achievement rate corresponding to the user and the completion rate corresponding to the user.

### BRIEF DESCRIPTION OF THE DRAWINGS

FIG. 1 is a flow chart of a training assessment method consistent with the present disclosure.

FIG. 2 is a flow chart showing determination of a completion rate corresponding to a user consistent with the present disclosure.

FIG. 3 is a schematic structural diagram of a training assessment device consistent with the present disclosure.

FIG. 4 is a schematic structural diagram of an electronic device consistent with the present disclosure.

### DETAILED DESCRIPTION OF THE EMBODIMENTS

When used in the present disclosure, the term “including” indicates the presence of the described features, wholes, steps, operations, elements and/or components, but does not exclude the presence or addition of one or more other features, entities, steps, operations, elements, components and/or their collections.

The term “and/or” used in the present disclosure refers to any combination of one or more of the items listed in association and all possible combinations, and includes these combinations.

As used in the specification and the claims in the present disclosure, the term “if” can be interpreted as “when . . . ” or “once” or “in response to determination” or “in response to detection” according to the context. Similarly, the phrase “if it is determined” or “if [the described condition or event] is detected” can be interpreted as meaning “once it is determined” or “in response to determination” or “once [the described condition or event] is detected” or “in response to detection of [the described condition or event]” according to the context.

In addition, in the specification and the claims in the present disclosure, the terms “first,” “second,” “third,” etc. are only used to distinguish the description, and should not be understood as indicating or implying relative importance.

References to “one embodiment” or “some embodiments” etc. described in the specification of the present disclosure mean that one or more embodiments of the present disclosure include specific features, structures or characteristics described in conjunction with the embodiments. Therefore, the statements “in one embodiment,” “in some embodiments,” “in some other embodiments,” etc., that appear in different places in this specification do not necessarily refer to the same embodiment, but mean “one or more but not all embodiments,” unless otherwise specifically emphasized in other ways. The terms “including,” “comprising,” “having” and their variations all mean “including but not limited to,” unless otherwise specifically emphasized in other ways. “Multiple” and “plurality of” mean “two or mor.”

Specific embodiments of the present disclosure are hereinafter described with reference to the accompanying drawings. The described embodiments are merely examples of the present disclosure and do not limit the scope of the present disclosure, which may be implemented in various ways. Specific structural and functional details described herein are not intended to limit, but merely serve as a basis for the claims and a representative basis for teaching one skilled in the art to variously employ the present disclosure in substantially any suitable detailed structure. The present disclosure may also be implemented in other embodiments without these specific details. In other cases, detailed descriptions of well-known systems, devices, circuits, and methods are omitted so as not to obscure the description of the present disclosure with unnecessary details.

The steps involved in the training assessment method provided in the embodiments of the present disclosure are only examples. Not all steps are steps that must be executed, and not all information or messages are required. Steps, information, and messages can be added or removed as needed during use. The same step or steps or messages with the same function in the embodiments of the present disclosure can be referenced to by each other in different embodiments.

The application scenario described in the embodiments of the present disclosure is used to more clearly illustrate the technical solution of the embodiments of the present disclosure, and does not constitute a limitation on the technical solution provided in the embodiments of the present disclosure. It is known to those of ordinary skill in the art that with the evolution of network architecture and the emergence of new application scenarios, the technical solution provided in the embodiments of the present disclosure is also applicable to similar technical problems.

During fitness training, it is needed to understand a user's training status, to assess the rationality of a training plan based on the training status, such that the training plan can be adjusted in a timely and effective manner to improve the training effect. A current training assessment method generally includes obtaining the amount of the training plan executed by the user (such as time and/or distance, etc.) to assess the user's training status based on the user's training amount. That is, the current training assessment method only performs assessment based on the training amount, and the assessment information is relatively unilateral and cannot fully reflect the user's training status, resulting in poor accuracy and effect of the training assessment.

The present disclosure provides a training assessment method and device, an electronic device, and a computer readable storage medium, to at least partially alleviate the above problem. In the training assessment method provided by the present disclosure, when it is needed to assess a user's training, motion data of the user when executing a preset training plan may be obtained, and an achievement rate and a completion rate corresponding to the user may be determined based on the motion data and the preset training plan, to determine assessment results of the user based on the achievement rate and the completion rate corresponding to the user. The achievement rate corresponding to the user may be used to characterize the user's achievement in terms of training intensity, and the completion rate corresponding to the user may be used to characterize the user's completion in terms of training volume. By combining training intensity and training volume to assess the user's training status, the comprehensiveness of the assessment information may be ensured, thereby improving the accuracy and effect of the training assessment.

In various embodiments of the present disclosure, the electronic device may be, for example, a mobile phone, a tablet computer, a wearable device, a notebook computer, an ultra-mobile personal computer (UMPC), a netbook, a personal digital assistant (PDA), a desktop computer, or a cloud server. The present disclosure does not limit the specific type of the electronic device.

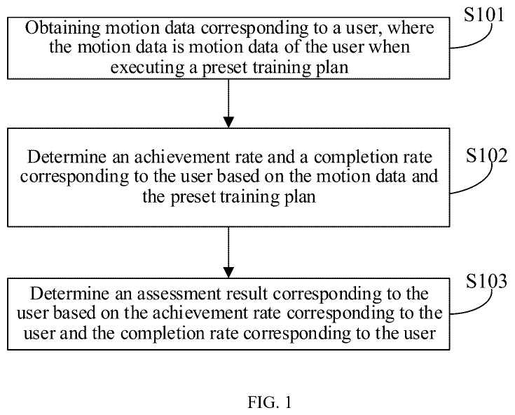

The present disclosure provides a training assessment method. In one embodiment, as shown in FIG. 1 which is a flow chart of an example training assessment method provided by the present disclosure, the training assessment method includes:

S101: obtaining motion data corresponding to a user, where the motion data is the motion data when the user executes a preset training plan;

S102: determining an achievement rate and a completion rate corresponding to the user based on the motion data and the preset training plan, where the achievement rate corresponding to the user is used to characterize the user's achievement in terms of training intensity and the completion rate corresponding to the user is used to characterize the user's completion of the training volume; and

S103: determining an assessment result corresponding to the user based on the achievement rate corresponding to the user and the completion rate corresponding to the user.

In various embodiments, the preset training plan may be a training plan formulated by the user, or a training plan formulated for the user by another user, such as a training plan formulated for the user by a fitness coach, etc. The present disclosure has no limit on this.

In one embodiment, when the user performs training according to the preset training plan, the user may wear a wearable device (such as a smart watch or smart glasses, etc.). The wearable device may collect the motion data of the user during training. Therefore, when it is needed to assess the user's training, an electronic device may obtain the corresponding motion data of the user from the wearable device to determine the assessment result of the user according to the corresponding motion data of the user.

Many types of fitness equipment have the function of collecting motion data. Therefore, in one embodiment, when the user is training according to the preset training plan and uses a fitness tool for training, the fitness tool may collect the motion data of the user during training. Therefore, when it is needed to assess the user's training, the electronic device may obtain the corresponding motion data of the user from the fitness tool to determine the assessment result of the user according to the corresponding motion data of the user.

In one embodiment, after the wearable device or fitness tool collects the motion data of the user during training, the motion data of the user during training may be sent to the cloud associated with the wearable device or fitness tool to save the motion data of the user during training through the cloud. Therefore, when it is needed to assess the user's training, the electronic device may obtain the corresponding motion data of the user from the cloud to determine the assessment result of the user according to the corresponding motion data of the user.

The above embodiments where the motion data of the user during training is collected through the wearable device or fitness tool are used as examples only to illustrate the present disclosure, and do not limit the scope of the present disclosure. The manner for collecting the motion data of the user during training is not limited in the present disclosure. For example, in some other embodiments, an application (such as an exercise application) that is able to collect the motion data may be installed in the electronic device. When the user is training according to the preset training plan, the user may start the exercise application in the electronic device and carry the electronic device to collect the motion data of the user during training through the exercise application in the electronic device. Therefore, when it is needed to assess the user's training, the electronic device may obtain the corresponding motion data of the user from the exercise application to determine the assessment result of the user according to the corresponding motion data of the user.

In one embodiment, the motion data may include one or more of pace, equivalent pace, power, heart rate, cadence, stride, cadence, time, distance or exercise load. For example, when the training content performed by the user is running, the motion data may include pace, equivalent pace, power, heart rate, cadence, stride, time, distance and exercise load. As another example, when the training content performed by the user is cycling, the motion data may include pace, equivalent pace, power, heart rate, cadence, time, distance and exercise load.

The above embodiments where the motion data includes one or more of pace, equivalent pace, power, heart rate, cadence, stride, cadence, time, distance or exercise load, etc., are used as examples only to illustrate the present disclosure, and do not limit the scope of the present disclosure. The motion data may be specifically determined according to the actual scenario.

Determination of the achievement rate corresponding to the user based on the motion data and the preset training plan in S102 will be described below in details.

In one embodiment, the preset training plan may include one or more training contents. Each training content may include one or more preset actions, and the one or more preset actions included in various training contents may be the same or different. Further, the preset training plan may also include an achievement standard corresponding to each preset action. For one same preset action, when multiple training contents all include one same preset action, the achievement standard corresponding to the preset action included in each training content may be the same or different.

For example, the preset training plan may include ten runs, and each run may include a warm-up action, a running action, a recovery action and a relaxation action. The preset training plan may also include the achievement standard corresponding to the warm-up action, the achievement standard corresponding to the running action, the achievement standard corresponding to the recovery action and the achievement standard corresponding to the relaxation action, in each run. That is, the preset training plan may also include: achievement standard A1 for the warm-up action, achievement standard B1 for the running action, achievement standard C1 for the recovery action, and achievement standard D1 for the relaxation action, in the first run; achievement standard A2 for the warm-up action, achievement standard B2 for the running action, achievement standard C2 for the recovery action, and achievement standard D2 for the relaxation action, in the second run; achievement standard A3 for the warm-up action, achievement standard B3 for the running action, achievement standard C3 for the recovery action, and achievement standard D3 for the relaxation action. in the third run; . . . ; and, achievement standard A10 for the warm-up action, achievement standard B10 for the running action, achievement standard C10 for the recovery action, and achievement standard D10 for the relaxation action, in the tenth run.

In another embodiment, the preset training plan may only include achievement standard A0 for the warm-up action, achievement standard B0 for the running action, achievement standard C0 for the recovery action, and achievement standard D0 for the relaxation action. That is, the achievement standards for the warm-up action in the first run, the second run, the third run, . . . , and the tenth run may all be A0; the achievement standards for the running action in the first run, the second run, the third run, . . . , and the tenth run may all be B0; the achievement standards for the recovery action in the first run, the second run, the third run, . . . , and the tenth run may all be C0; and the achievement standards for the relaxation action in the first run, the second run, the third run, . . . , and the tenth run may all be D0.

The motion data corresponding to the user obtained by the electronic device may include the motion data when the user executes one training content, or may include the motion data when the user executes the multiple training contents. For example, when the preset training plan includes ten runs, the motion data corresponding to the user obtained by the electronic device may include the motion data when the user runs for the first time or include the motion data when the user runs for the second time. Or, the motion data corresponding to the user obtained by the electronic device may include the motion data when the user runs for the first time, the motion data when the user runs for the second time, and the motion data when the user runs for the third time, and so on.

When the motion data corresponding to the user obtained by the electronic device includes the motion data when the user executes one training content, the achievement rate corresponding to the user may be the achievement rate when the user executes the one training content. When the motion data corresponding to the user obtained by the electronic device includes the motion data when the user executes the multiple training contents, the corresponding achievement rate of the user may be the comprehensive achievement rate when the user executes the multiple training contents.

In one embodiment, when the motion data corresponding to the user obtained by the electronic device includes the motion data when the user executes one training content, the electronic device may determine one or more preset actions included in the training content. Subsequently, for each preset action, the electronic device may determine the achievement standard corresponding to the preset action according to the preset training plan, and may determine the achievement rate corresponding to the preset action according to the motion data and the achievement standard corresponding to the preset action, to determine the achievement rate corresponding to the user based on the achievement rate corresponding to each preset action in the training content, that is, determine the achievement rate corresponding to the user when executing the training content this time.

In another embodiment, when the motion data corresponding to the user obtained by the electronic device includes the motion data when the user executes the multiple training contents, the electronic device may determine the motion data corresponding to each training content according to the motion data corresponding to the user. For each training content, the electronic device may determine one or more preset actions included in the training content. Subsequently, for each preset action in each time of executing one training content, the electronic device may determine the achievement standard corresponding to the preset action according to the preset training plan, and may determine the achievement rate corresponding to the preset action according to the motion data corresponding to the training content and the achievement standard corresponding to the preset action. For each training content, the electronic device may determine the achievement rate when the user performs the training content according to the achievement rate corresponding to each preset action in the training content, such that the achievement rate corresponding to the user may be determined according to the achievement rate when the user performs each training content.

In one embodiment, after determining the achievement rate when the user performs each training content (hereinafter referred to as achievement rate A), the electronic device may determine the average value of the achievement rate A, and may determine the average value of the achievement rate A as the achievement rate corresponding to the user. In another embodiment, the electronic device may obtain an achievement weight corresponding to each training content, and may perform weighted averaging on the achievement rate A according to the achievement weights corresponding to each training content to obtain the achievement rate corresponding to the user.

In one embodiment, the electronic device may determine the achievement rate when the user executes each training content according to:

$${SR}_{j} = {\sum }_{i = 1}^{i = n} ( {AC}_{ji} * {WA}_{ji} ) ;$$

where SRⱼ is the achievement rate when the user executes the j-th training content, ACⱼᵢ is the achievement rate corresponding to the i-th preset action of the j-th training content, WAⱼᵢ is the achievement weight corresponding to the i-th preset action of the j-th training content, and n is the number of the preset actions in the j-th training content.

For each training content, the achievement weight corresponding to each preset action of the training content may be the same or different, and the present disclosure has no limit on this, which may be specifically determined according to actual conditions. The achievement weight corresponding to each preset action may be set by the user, or may be determined by default by the electronic device.

For example, when a certain training content includes a warm-up action, a running action, a recovery action, and a relaxation action, the electronic device may default to determine that the achievement weight corresponding to the warm-up action, the achievement weight corresponding to the running action, the achievement weight corresponding to the recovery action, and the achievement weight corresponding to the relaxation action are all 25%. As another example, the electronic device may default to determine that the achievement weight corresponding to the warm-up action are 20%, the achievement weight corresponding to the running action are 30%, the achievement weight corresponding to the recovery action are 25%, and the achievement weight corresponding to the relaxation action are 25%, etc.

Determining the achievement rate of one action according to the motion data and the achievement standard of the action will be described below.

In one embodiment, for each preset action, the electronic device may determine achievement actions according to the motion data and the achievement standard corresponding to the preset action, such that the electronic device may determine the achievement rate corresponding to the preset action according to the achievement action.

Each preset action may generally have a corresponding training duration. That is, the motion data may include the motion data when the user performs the preset action within the training duration. Therefore, the electronic device may determine the motion data when the user performs the preset action each time according to the motion data within the training duration, and determine whether the preset action performed by the user each time meets the standard according to the motion data when the user performs the preset action each time and the achievement standard corresponding to the preset action. When it is determined that the motion data when the user performs the preset action at a certain time meets the achievement standard corresponding to the preset action, the electronic device may determine that the preset action performed by the user this time meets the standard, and may determine the preset action performed by the user this time as one achievement action. Subsequently, the electronic device may determine the achievement rate corresponding to the preset action according to all achievement actions.

In some embodiments, the electronic device may determine the one execution of the preset action according to the frequency of collecting motion data. For example, when the frequency of motion data collection is 60 times per minute, that is, when motion data is collected 60 times in one minute, the electronic device may determine one second as one execution, that is, the preset action executed every second may be one execution of the preset action. As another example, when the frequency of motion data collection is 6 times per minute, that is, when motion data is collected 6 times in one minute, the electronic device may determine ten seconds as one execution, that is, the preset action executed every ten seconds may be one execution of the preset action. As another example, when the frequency of motion data collection is once per minute, that is, when motion data is collected once in one minute, one minute may be determined as one execution, that is, the preset action executed every minute may be one execution of the preset action, and so on. In the following examples, the preset action executed in one minute is determined as one execution of the preset action.

For each preset action, the preset training plan may include an intensity interval of the achievement standard corresponding to the preset action (hereinafter referred to as the standard intensity interval). Therefore, when it is needed to determine whether a preset action executed at a certain time is one achievement action, the electronic device may obtain the standard intensity interval corresponding to the preset action according to the preset training plan, and then determine the actual intensity corresponding to the preset action executed at this time according to the motion data corresponding to the preset action executed at this time, that is, determine the intensity when the user executes the preset action, to determine whether the preset action executed at this time meets the standard according to the actual intensity corresponding to the preset action executed at this time and the standard intensity interval. When the actual intensity corresponding to the preset action executed at this time is within the standard intensity interval, it may be determined that the preset action executed at this time meets the standard and the electronic device may determine the preset action executed at this time as one achievement action.

For example, for each preset action, after determining the corresponding achievement actions, the electronic device may obtain the first duration corresponding to the achievement actions, that is, obtain the sum of the durations corresponding to all achievement actions. Also, the electronic device may determine the second duration corresponding to the preset action according to the preset training plan, and the second duration may be the duration during which the intensity of the preset action specified in the preset training plan needs to be within the standard intensity range. Subsequently, the electronic device may determine the achievement rate corresponding to the preset action according to the first duration corresponding to the standard achievement actions and the second duration corresponding to the preset action. In some embodiments, the electronic device may determine the ratio between the first duration and the second duration as the achievement rate corresponding to the preset action.

For example, when the training content is running and running includes a warm-up action, a running action, a recovery action, and a relaxation action, it may be assumed that the preset training plan stipulates that the training duration of the warm-up action must be 5 minutes, and the heart rate corresponding to the 5-minute warm-up action must be between 122 bpm and 141 bpm. That is, the achievement standard corresponding to the warm-up action may be the heart rate, and the standard intensity interval corresponding to the heart rate may be [122 bpm, 141 bpm]. At this time, after the electronic device obtains the motion data of the user when running, it may determine the motion data corresponding to the warm-up action according to the motion data of the user when running. For example, it may be determined that the motion data of the first 4 minutes from the motion data of running is the motion data corresponding to the warm-up action. Subsequently, the electronic device may determine the motion data corresponding to each minute of the warm-up action, that is, determine the heart rate corresponding to each minute of the warm-up action, and may determine whether each heart rate is within [122 bpm, 141 bpm].

Assuming that the heart rate corresponding to the first minute of warm-up is 114 bpm, the heart rate corresponding to the second minute of warm-up is 130 bpm, the heart rate corresponding to the third minute of warm-up is 125 bpm, and the heart rate corresponding to the fourth minute of warm-up is 120 bpm, the electronic device may determine that the heart rate corresponding to the second minute of warm-up and the heart rate corresponding to the third minute of warm-up are within [122 bpm, 141 bpm]. Therefore, the electronic device may determine that the second minute of warm-up and the third minute of warm-up are standard achievement actions, and may determine that the first duration corresponding to the standard achievement actions is 2 minutes. At the same time, the electronic device may determine that the second duration corresponding to the warm-up is 5 minutes according to the preset training plan. Therefore, the electronic device may determine that the achievement rate corresponding to the warm-up is 2/5=40%.

In one embodiment, there may be a plurality of achievement standards for each preset action. When one preset action (for example, preset action A) corresponds to a plurality of achievement standards, the electronic device may determine the motion data corresponding to preset action A according to the motion data corresponding to the user, and then determine the motion data related to each of the plurality of achievement standards according to the motion data corresponding to preset action A. Then, the electronic device may determine the achievement rates of the preset action on each of the plurality of achievement standards according to the motion data related to each of the plurality of achievement standards. Subsequently, the electronic device may determine the achievement rate corresponding to the preset action according to the achievement rate of the preset action on each of the plurality of achievement standards and the standard achievement weight corresponding to each of the plurality of achievement standards.

The standard achievement weight corresponding to each of the plurality of achievement standards may be specifically determined according to the actual scenario, and the present disclosure has no limit on this.

For example, in one embodiment, in the running action corresponding to running, when the achievement standards corresponding to the running action includes pace and cadence, the weight corresponding to the pace may be 70%, and the weight corresponding to the cadence may be 30%. After the electronic device obtains the corresponding motion data of the user, it may determine the motion data corresponding to the running action according to the corresponding motion data of the user, and then determine the motion data related to the pace and the motion data related to the cadence according to the motion data corresponding to the running action, that is, the pace and cadence of the user when performing the running action may be determined respectively according to the motion data corresponding to the running action.

Then, the electronic device may determine the achievement rate of the running action performed by the user in terms of the pace (e.g., achievement rate A) based on the pace of the user when performing the running action and the standard intensity interval corresponding to the pace specified in the preset training plan. At the same time, the electronic device may determine the achievement rate of the running action performed by the user in terms of the step frequency (e.g., achievement rate B) based on the step frequency of the user when performing the running action and the standard intensity interval corresponding to the step frequency specified in the preset training plan. Subsequently, the electronic device may determine the achievement rate corresponding to the running action based on achievement rate A and achievement rate B, that is, the achievement rate corresponding to the running action may be (achievement rate A*70%+achievement rate B*30%).

Determination of the completion rate corresponding to the user according to the motion data and the preset training plan in S102 will be described in detail below.

In one embodiment, the preset training plan may include one or more training contents. Each training content may include one or more preset actions. The preset training plan may also include the completion standards corresponding to each preset action. For one same preset action, when multiple training contents all include the preset action, the completion standards corresponding to the preset actions included in each training content may be the same or different.

For example, the preset training plan may include ten runs, and each run may include a warm-up action, a running action, a recovery action and a relaxation action. At this time, the preset training plan may also include the completion standard corresponding to the warm-up action, the completion standard corresponding to the running action, the completion standard corresponding to the recovery action and the completion standard corresponding to the relaxation action in each run. That is, the preset training plan may also include: in the first run, completion standard E1 corresponding to the warm-up action, completion standard F1 corresponding to the running action, completion standard G1 corresponding to the recovery action, and completion standard H1 corresponding to the relaxation action; in the second run, completion standard E2 corresponding to the warm-up action, completion standard F2 corresponding to the running action, completion standard G2 corresponding to the recovery action, and completion standard H2 corresponding to the relaxation action; in the third run, completion standard E3 corresponding to the warm-up action, completion standard F3 corresponding to the running action, completion standard G3 corresponding to the recovery action, and completion standard H3 corresponding to the relaxation action; . . . ; and in the tenth run, completion standard E10 corresponding to the warm-up action, completion standard F10 corresponding to the running action, completion standard G10 corresponding to the recovery action, and completion standard H10 corresponding to the relaxation action.

As another example, the preset the training plan may only include completion standard E0 corresponding to the warm-up action, completion standard F0 corresponding to the running action, completion standard G0 corresponding to the recovery action, and completion standard H0 corresponding to the relaxation action. That is, in the first run, the second run, the third run, . . . , and the tenth run, the completion standards corresponding to the warm-up action may all be E0. In the first run, the second run, the third run, . . . , and the tenth run, the completion standards corresponding to the running action may all be F0. In the first run, the second run, the third run, . . . , and the tenth run, the completion standards corresponding to the recovery action may all be G0. In the first run, the second run, the third run, . . . , and the tenth run, the completion standards corresponding to the relaxation action may all be H0.

The motion data corresponding to the user obtained by the electronic device may include the motion data when the user executes one training content, or may include the motion data when the user executes the multiple training contents. For example, when the preset training plan includes ten runs, the motion data corresponding to the user obtained by the electronic device may include the motion data when the user runs for the first time or include the motion data when the user runs for the second time. Or, the motion data corresponding to the user obtained by the electronic device may include the motion data when the user runs for the first time, the motion data when the user runs for the second time, and the motion data when the user runs for the third time, and so on.

When the motion data corresponding to the user obtained by the electronic device includes the motion data when the user executes one training content, the corresponding completion rate of the user may be the completion rate when the user executes the one training content. When the motion data corresponding to the user obtained by the electronic device includes the motion data when the user executes the multiple training contents, the corresponding completion rate of the user may be the comprehensive completion rate when the user executes the multiple training contents.

In one embodiment, when the motion data corresponding to the user obtained by the electronic device includes the motion data when the user executes one training content, the electronic device may determine one or more preset actions included in the training content. Subsequently, for each preset action, the electronic device may determine the completion standard corresponding to the preset action according to the preset training plan, and may determine the completion rate corresponding to the preset action according to the motion data and the completion standard corresponding to the preset action, to determine the user's corresponding completion rate based on the completion rates corresponding to each preset action in the training content, that is, determine the completion rate of the user when executing the training content this time.

In another embodiment, when the motion data corresponding to the user obtained by the electronic device includes the motion data when the user executes the multiple training contents, the electronic device may determine the motion data corresponding to each training content according to the motion data corresponding to the user. For each training content, the electronic device may determine one or more preset actions included in the training content. Subsequently, for each preset action in each time of executing one training content, the electronic device may determine the completion standard corresponding to the preset action according to the preset training plan, and may determine the completion rate corresponding to the preset action according to the motion data corresponding to the training content and the completion standard corresponding to the preset action. For each training content, the electronic device may determine the completion rate when the user performs the training content according to the completion rate corresponding to each preset action in the training content, such that the completion rate corresponding to the user may be determined according to the completion rate when the user performs each training content.

In one embodiment, after determining the completion rate when the user performs each training content (hereinafter referred to as completion rate B), the electronic device may determine the average value of completion rate B, and may determine the average value of the completion rate B as the completion rate corresponding to the user. In another embodiment, the electronic device may obtain a completion weight corresponding to each training content, and may perform weighted averaging on completion rate B according to the completion weights corresponding to each training content to obtain the completion rate corresponding to the user.

In one embodiment, the electronic device may determine the completion rate when the user executes each training content according to:

$${CR}_{j} = {\sum }_{i = 1}^{i = n} ( {BC}_{ji} * {WB}_{ji} ) ;$$

where CRⱼ is the completion rate when the user executes the j-th training content, BCⱼᵢ is the completion rate corresponding to the i-th preset action of the j-th training content, WBⱼᵢ is the completion weight corresponding to the i-th preset action of the j-th training content, and n is the number of the preset actions in the j-th training content.

For each training content, the completion weight corresponding to each preset action of the training content may be the same or different, and the present disclosure has no limit on this, which may be specifically determined according to actual conditions. The completion weight corresponding to each preset action may be set by the user, or may be determined by default by the electronic device.

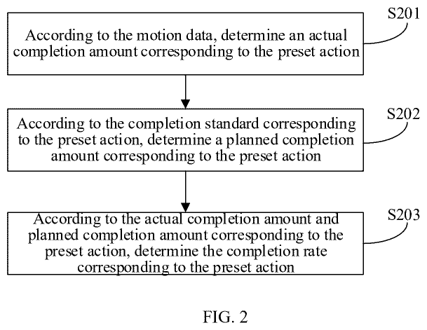

In one embodiment shown in FIG. 2 illustrating a flow chart for determining the completion rate corresponding to the user, determining a completion rate corresponding to one preset action according to the motion data and the completion standard of the preset action may include:

S201: according to the motion data, determining the actual completion amount corresponding to the preset action;

S202: according to the completion standard corresponding to the preset action, determining the planned completion amount corresponding to the preset action; and

S203: according to the actual completion amount and the planned completion amount corresponding to the preset action, determining the completion rate corresponding to the preset action.

For each preset action, the electronic device may determine the actual completion amount corresponding to the preset action according to the motion data. Subsequently, the electronic device may determine the planned completion amount corresponding to the preset action according to the completion standard corresponding to the preset action, to determine the completion rate corresponding to the preset action according to the actual completion amount and the planned completion amount corresponding to the preset action.

For example, when the training content is running, and running includes a warm-up action, a running action, a recovery action and a relaxation action, it may be assumed that the time for the running action specified in the preset training plan is 25 minutes. That is, the completion standard corresponding to the running action is time, and the planned completion amount corresponding to the time is 25 minutes. At this time, the electronic device may obtain the motion data of the user when running, and may determine the motion data corresponding to the running action according to the motion data of the user when running. For example, the motion data from the 5th minute to the 22nd minute may be determined from the motion data when running as the motion data corresponding to the running action. Subsequently, the electronic device may determine the actual completion amount of the running action according to the motion data corresponding to the running action, that is, the actual completion amount of the running action may be determined to be 18 minutes. Therefore, the electronic device may determine the completion rate of the running action according to the actual completion amount and the planned completion amount corresponding to the running action, that is, the completion rate of the running action may be determined to be 18/25=72%.

In one embodiment, there may be a plurality of completion standards for each preset action. When one preset action (for example, a preset action A) corresponds to a plurality of completion standards, the electronic device may determine the motion data corresponding to the preset action A according to the motion data corresponding to the user, and then determine the motion data related to each of the plurality of completion standards according to the motion data corresponding to the preset action A. Then, the electronic device may determine the completion rates of the preset action on each of the plurality of completion standards according to the motion data related to each of the plurality of completion standards. Subsequently, the electronic device may determine the completion rate corresponding to the preset action according to the completion rate of the preset action on each of the plurality of completion standards and the standard achievement weight corresponding to each of the plurality of completion standards.

The completion weight corresponding to each of the plurality of completion standards may be determined according to actual application scenarios, and the present disclosure has no limit on this.

In one embodiment, after determining the achievement rate and the completion rate corresponding to the user, the electronic device may determine the assessment result corresponding to the user according to:

$$Score = 100 * ( SR * WS + CR * WC ) ,$$

where Score is the assessment result corresponding to the user, SR is the achievement rate corresponding to the user, WS is the assessment weight corresponding to the achievement rate, CR is the completion rate corresponding to the user, and WC is the assessment weight corresponding to the completion rate.

When the assessment result is higher, the execution of the user's training plan may be better. When the assessment result is lower, the execution of the user's training plan may be worse. In one embodiment, after determining the assessment result corresponding to the user, the assessment result corresponding to the user and the exercise data corresponding to the user may be displayed in the electronic device, to facilitate the user or a fitness coach to understand the execution of the user's training plan and reasonably adjust the training plan according to the execution of the user's training plan.

For example, when the assessment result corresponding to the user is poor, such as lower than the preset assessment threshold, the user or the fitness coach may view the user's training details information, and the training details information may include the user's corresponding achievement rate and completion rate. That is, the user may view the user's corresponding achievement rate and completion rate in the electronic device. When it is determined that the poor assessment result corresponding to the user is caused by the low achievement rate corresponding to the user, the user or the fitness coach may reasonably adjust the intensity of the preset training plan according to the actual situation of the user. When it is determined that the poor assessment result corresponding to the user is caused by the low completion rate corresponding to the user, the user or the fitness coach may reasonably adjust the completion amount of the preset training plan according to the actual situation of the user. Therefore, the user may be able to complete the preset training plan, thereby gradually improving the user's exercise level.

The preset assessment threshold may be set by the user or by the fitness coach, and the present embodiment has no limit on this. For example, the fitness coach may set the preset assessment threshold to any value such as 50 points or 60 points according to the actual training status.

In the present disclosure, when it is needed to assess the user's training, the motion data of the user when executing the preset training plan may be obtained, and the achievement rate and completion rate corresponding to the user may be determined based on the motion data and the preset training plan, to determine the user's corresponding assessment result based on the achievement rate and completion rate corresponding to the user. The achievement rate corresponding to the user may be used to characterize the user's achievement with the training intensity, and the completion rate corresponding to the user may be used to characterize the user's completion amount of the training. The user's training status may be assessed by combining the training intensity and the training amount, ensuring the comprehensiveness of the assessment information and thereby improving the accuracy and effect of the training assessment.

The sequence number of each step in the above embodiments does not mean the order of execution. The execution order of each process should be determined by its function and internal logic, and should not constitute any limitation on the implementation process of the embodiment of the present disclosure.

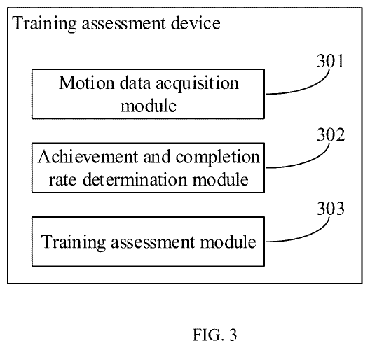

The present disclosure also provides a training assessment device. In one embodiment shown in FIG. 3 which is a schematic structural diagram of an example training assessment device provided by the present disclosure, the device may include:
a motion data acquisition module 301, configured to obtain motion data corresponding to a user, where the motion data is motion data when the user executes a preset training plan;
an achievement and completion rate determination module 302, configured to determine an achievement rate and a completion rate corresponding to the user according to the motion data and the preset training plan, where the achievement rate corresponding to the user may be used to characterize the user's achievement with the training intensity, and the completion rate corresponding to the user may be used to characterize the user's completion amount of the training in terms of training volume; and
a training evaluation module 303, configured to determine the evaluation result corresponding to the user according to the achievement rate corresponding to the user and the completion rate corresponding to the user.

In one embodiment, the preset training plan may include achievement standards corresponding to one or more preset actions.

Accordingly, the achievement and completion rate determination module 302 may also include:
a first achievement rate determination unit, configured to determine the achievement rate corresponding to each of the one or more preset actions according to the motion data and the achievement standard corresponding to the preset action; and
a second achievement rate determination unit, configured to determine the achievement rate corresponding to the user according to the achievement rate corresponding to each of the one or more preset actions.

In another embodiment, the preset training plan may include completion standards corresponding to one or more preset actions.

Accordingly, the achievement and completion rate determination module 302 may also include:
a first completion rate determination unit, used to determine the completion rate corresponding to each of the one or more preset actions based on the motion data and the completion standard corresponding to the preset action; and
a second completion rate determination unit, used to determine the completion rate corresponding to the user based on the completion rate corresponding to each of the preset actions.

In one embodiment, the first achievement rate determination unit may be further used to determine achievement actions based on the motion data and the achievement standards corresponding to one preset action, where one achievement action may be an action among the preset actions that meets the achievement standard corresponding to the preset action; and to determine the achievement rate corresponding to the preset action based on the achievement actions.

In one embodiment, the first achievement rate determination unit may be further used to obtain a standard intensity interval of the achievement standard corresponding to one preset action; determine the intensity corresponding to the preset action based on the motion data; and determine the achievement actions based on the intensity corresponding to the preset action and the standard intensity interval.

In one embodiment, the first achievement rate determination unit may be further used to obtain a first duration corresponding to one achievement action and a second duration corresponding to the preset action, where the second duration is the duration during which the intensity of the preset action specified in the preset training plan is within the standard intensity interval; and determine the achievement rate corresponding to the preset action based on the first duration and the second duration.

In another embodiment, the first completion rate determination unit may be further used to determine the actual completion amount corresponding to the preset action according to the motion data; determine the planned completion amount corresponding to the preset action according to the completion standard corresponding to the preset action; and determine the completion rate corresponding to the preset action according to the actual completion amount and the planned completion amount corresponding to the preset action.

The information interaction, execution process, etc. between the above-mentioned devices/units/modules are based on the same concept as the method embodiments of the present disclosure, and their specific functions and technical effects can be found in the method embodiment section, which will not be repeated here.

Those skilled in the art can clearly understand that for the convenience and simplicity of description, only the division of the above-mentioned functional units and modules is used as an example for illustration. In practical applications, the above-mentioned function allocation can be completed by different functional units and modules as needed, that is, the internal structure of the device can be divided into different functional units or modules to complete all or part of the functions described above. The functional units and modules in the embodiment can be integrated in a processing unit, or each unit can exist physically separately, or two or more units can be integrated in one unit. The above-mentioned integrated unit can be implemented in the form of hardware or in the form of software functional units. In addition, the specific names of the functional units and modules are only for the convenience of distinguishing each other, and are not used to limit the scope of the present disclosure. The specific working process of the units and modules in the above-mentioned system can refer to the corresponding process in the aforementioned method embodiments, which will not be repeated here.

The present disclosure also provides an electronic device. The electronic device may be a mobile phone, a tablet computer, a wearable device, a laptop computer, an ultra-mobile personal computer (UMPC), a netbook, a personal digital assistant (PDA), a desktop computer, a cloud server, etc.

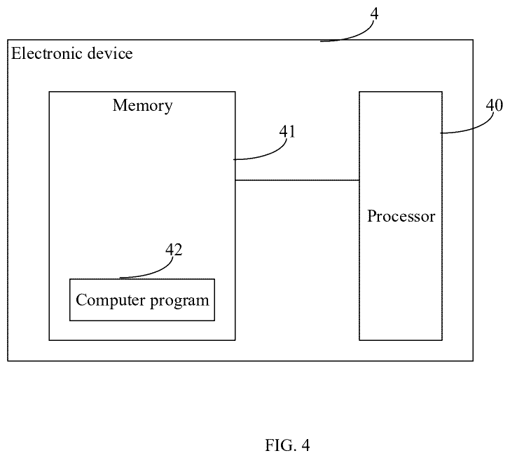

As shown in FIG. 4, in one embodiment, the electronic device 13 may include at least one processor 40 (only one is shown in the drawings) and at least one memory 41 (only one is shown in the drawings) storing at least one computer program 42. The at least one computer program 42 can be executed in the at least one processor 40. The at least one processor 40 may execute the at least one computer program 42, to implement any training assessment method consistent with the present disclosure.

In one embodiment, the electronic device 4 may include, but is not limited to, a processor 40 and a memory 41. The embodiment shown in FIG. 4 is only used as an example to illustrate the present disclosure, and does not limit the scope of the present disclosure. The electronic device may include more or fewer components than shown in the figure, or combine certain components, or different components, such as input and output devices, network access devices, etc.

In various embodiments, the processor 40 may be a central processing unit (CPU), and may also be other general-purpose processors, digital signal processors (DSP), application-specific integrated circuits (ASIC), field-programmable gate arrays (FPGA) or other programmable logic devices, discrete gates or transistor logic devices, discrete hardware components, etc. The general-purpose processor may be a microprocessor or any conventional processor, etc.

In some embodiments, the memory may be an internal storage unit of the electronic device, such as a hard disk or memory of the terminal device. In some other embodiments, the memory may also be an external storage device of the electronic device, for example, a plug-in hard disk, a smart media card (SMC), a secure digital (SD) card, a flash card, etc. equipped on the terminal device. The memory may also include both an internal storage unit of the electronic device and an external storage device. The memory may be used to store an operating system, an application program, a boot loader, data, or other programs, such as program codes of a computer program. The memory may also be used to temporarily store data that has been output or is to be output.

The present disclosure also provides a computer readable storage medium. The computer readable storage medium may be configured to store a computer program. When the computer program is executed by a processor, the training assessment method provided by various embodiments of the present disclosure may be implemented.

The present disclosure also provides a computer program product. When the computer program product is executed on an electronic device, the training assessment method provided by various embodiments of the present disclosure may be implemented through the electronic device.

All or part of the processes in the methods provided by various embodiments of the present disclosure may be implemented by instructing the relevant hardware through a computer program. The computer program may be stored in a computer-readable storage medium. When the computer program is executed by a processor, the steps of each of the above-mentioned method embodiments may be implemented. The computer program may include computer program codes, and the computer program codes may be in a source code form, an object code form, an executable file or some intermediate forms. The computer-readable medium may at least include: any entity or device that is able to carry the computer program codes to the terminal device, a recording medium, a computer memory, a read-only memory (ROM), a random access memory (RAM), an electrical carrier signal, a telecommunication signal, or a software distribution medium such as, for example, a USB flash drive, a mobile hard disk, a disk or an optical disk.

Each embodiment in this specification is described in a progressive mode, and each embodiment focuses on the difference from other embodiments. Same and similar parts of each embodiment may be referred to each other. As for the device disclosed in the embodiments, since it corresponds to the method disclosed in the embodiments, the description is relatively simple, and for relevant details, the reference may be made to the description of the method embodiments.

Devices and algorithm steps of the examples described in conjunction with the embodiments disclosed herein may be implemented by electronic hardware, computer software or a combination of the two. To clearly illustrate the possible interchangeability between the hardware and software, in the above description, the composition and steps of each example have been generally described according to their functions. Whether these functions are executed by hardware or software depends on the specific application and design constraints of the technical solution. Those skilled in the art may use different methods to implement the described functions for each specific application, but such implementation should not be regarded as exceeding the scope of the present disclosure.

In the present disclosure, the drawings and descriptions of the embodiments are illustrative and not restrictive. The same drawing reference numerals identify the same structures throughout the description of the embodiments. In addition, figures may exaggerate the thickness of some layers, films, screens, areas, etc., for purposes of understanding and ease of description. When an element such as a layer, film, region or substrate is referred to as being “on” another element, it may be directly on the another element or intervening elements may be present. In addition, “on” refers to positioning an element on or below another element, but does not essentially mean positioning on the upper side of another element according to the direction of gravity.

The orientation or positional relationship indicated by the terms “upper,” “lower,” “top,” “bottom,” “inner,” “outer,” etc. are based on the orientation or positional relationship shown in the drawings, and are only for the convenience of describing the present disclosure, rather than indicating or implying that the device or element referred to must have a specific orientation, be constructed and operated in a specific orientation, and therefore cannot be construed as a limitation of the present disclosure. When a component is said to be “connected” to another component, it may be directly connected to the other component or there may be an intermediate component present at the same time.

It should also be noted that in this article, relational terms such as “first” and “second” are only used to distinguish one entity or operation from another entity or operation, and do not necessarily require or imply that there is such actual relationship or sequence between these entities or operations them. Furthermore, the terms “comprises,” “includes,” or any other variation thereof are intended to cover a non-exclusive inclusion, such that an article or device including a list of elements includes not only those elements, but also other elements not expressly listed. Or it also includes elements inherent to the article or equipment. Without further limitation, an element defined by the statement “comprises a . . . ” does not exclude the presence of other identical elements in an article or device that includes the above-mentioned element.

The disclosed equipment and methods may be implemented in other ways. The device embodiments described above are only illustrative. For example, the division of the units is only a logical function division. In actual implementation, there may be other division methods, such as: multiple units or components may be combined, or can be integrated into another system, or some features can be ignored, or not implemented. In addition, the coupling, direct coupling, or communication connection between the components shown or discussed may be through some interfaces, and the indirect coupling or communication connection of the devices or units may be electrical, mechanical, or other forms.

The units described above as separate components may or may not be physically separated. The components shown as units may or may not be physical units. They may be located in one place or distributed to multiple network units. Some or all of the units may be selected according to actual needs to achieve the purpose of the solution of the present disclosure.

In addition, all functional units in the embodiments of the present disclosure may be integrated into one processing unit, or each unit may be separately used as a unit, or two or more units can be integrated into one unit. The above-mentioned integration units can be implemented in the form of hardware or in the form of hardware plus software functional units.

Various embodiments have been described to illustrate the operation principles and example implementations. The present disclosure is not limited to the specific embodiments described herein and that various other obvious changes, rearrangements, and substitutions will occur to those skilled in the art without departing from the scope of the present disclosure. Thus, while the present disclosure has been described in detail with reference to the above described embodiments, the present disclosure is not limited to the above described embodiments, but may be embodied in other equivalent forms without departing from the scope of the present disclosure.

1. A training assessment method comprising:
controlling a wearable device to collect motion data of a user when executing a preset training plan;
determining an achievement rate and a completion rate corresponding to the user based on the motion data from the wearable device and the preset training plan, the achievement rate corresponding to the user characterizing an achievement situation of the user in terms of training intensity, and the completion rate corresponding to the user characterizing a completion situation of the user in terms of training volume;
determining an assessment result based on the achievement rate corresponding to the user and the completion rate corresponding to the user; and
controlling an electronic device to generate an output according to the assessment result.

2. The method according to claim 1, wherein:
the preset training plan includes an achievement standard corresponding to a preset action; and
determining the achievement rate and the completion rate corresponding to the user according to the motion data and the preset training plan includes:
determining an achievement rate corresponding to the preset action according to the motion data and the action achievement standard corresponding to the preset action; and
determining the achievement rate corresponding to the user according to the achievement rate corresponding to the preset action.

3. The method according to claim 2, wherein determining the achievement rate corresponding to the preset action according to the motion data and the achievement standard corresponding to the preset action includes:
determining an achievement action meeting the achievement standard corresponding to the preset action according to the motion data and the achievement standard corresponding to the preset action; and
determining the achievement rate corresponding to the preset action according to the achievement action.

4. The method according to claim 3, wherein determining the achievement action according to the motion data and the achievement standard corresponding to the preset action includes:
obtaining a standard intensity interval of the achievement standard corresponding to the preset action;
determining an intensity corresponding to the preset action according to the motion data; and
determining the achievement action according to the intensity corresponding to the preset action and the standard intensity interval.

5. The method according to claim 4, wherein determining the achievement rate corresponding to the preset action according to the achievement action includes:
obtaining a first duration corresponding to the achievement action and a second duration corresponding to the preset action, the second duration being a duration in which the intensity of the preset action specified in the preset training plan is within the standard intensity interval; and
determining the achievement rate corresponding to the preset action according to the first duration and the second duration.

6. The method according to claim 1, wherein:
the preset training plan includes a completion standard corresponding to a preset action; and
determining the achievement rate and the completion rate corresponding to the user based on the motion data and the preset training plan includes:
determining a completion rate corresponding to the preset action based on the motion data and the completion standard corresponding to the preset action; and
determining the completion rate corresponding to the user based on the completion rate corresponding to the preset action.

7. The method according to claim 6, wherein determining the completion rate corresponding to the preset action based on the motion data and the completion standard corresponding to the preset action includes:
determining an actual completion amount corresponding to the preset action according to the motion data;
determining a planned completion amount corresponding to the preset action according to the completion standard corresponding to the preset action; and
determining the completion rate corresponding to the preset action according to the actual completion amount and the planned completion amount corresponding to the preset action.

8. A wearable device comprising:
at least one memory storing at least one computer program; and
at least one processor configured to execute the at least one computer program to:
control the wearable device to collect motion data of a user when executing a preset training plan;
determine an achievement rate and a completion rate corresponding to the user based on the motion data from the wearable device and the preset training plan, the achievement rate corresponding to the user characterizing an achievement situation of the user in terms of training intensity, and the completion rate corresponding to the user characterizing a completion situation of the user in terms of training volume;
determine an assessment result based on the achievement rate corresponding to the user and the completion rate corresponding to the user; and
control the wearable device to generate an output according to the assessment result.

9. The device according to claim 8, wherein:
the preset training plan includes an achievement standard corresponding to a preset action; and
the at least one processor is further configured to execute the at least one computer program to:
determine an achievement rate corresponding to the preset action according to the motion data and the action achievement standard corresponding to the preset action; and
determine the achievement rate corresponding to the user according to the achievement rate corresponding to the preset action.

10. The device according to claim 9, wherein the at least one processor is further configured to execute the at least one computer program to:
determine an achievement action meeting the achievement standard corresponding to the preset action according to the motion data and the achievement standard corresponding to the preset action; and
determine the achievement rate corresponding to the preset action according to the achievement action.

11. The device according to claim 10, wherein the at least one processor is further configured to execute the at least one computer program to:
obtain a standard intensity interval of the achievement standard corresponding to the preset action;
determine an intensity corresponding to the preset action according to the motion data; and
determine the achievement action according to the intensity corresponding to the preset action and the standard intensity interval.

12. The device according to claim 10, wherein the at least one processor is further configured to execute the at least one computer program to:
obtain a first duration corresponding to the achievement action and a second duration corresponding to the preset action, the second duration being a duration in which the intensity of the preset action specified in the preset training plan is within the standard intensity interval; and
determine the achievement rate corresponding to the preset action according to the first duration and the second duration.

13. The device according to claim 9, wherein:
the preset training plan includes a completion standard corresponding to a preset action; and
the at least one processor is further configured to execute the at least one computer program to:
determine a completion rate corresponding to the preset action based on the motion data and the completion standard corresponding to the preset action; and
determine the completion rate corresponding to the user based on the completion rate corresponding to the preset action.

14. The device according to claim 13, wherein the at least one processor is further configured to execute the at least one computer program to:
determine an actual completion amount corresponding to the preset action according to the motion data;
determine a planned completion amount corresponding to the preset action according to the completion standard corresponding to the preset action; and
determine the completion rate corresponding to the preset action according to the actual completion amount and the planned completion amount corresponding to the preset action.

15. A non-transitory computer readable storage medium storing at least one computer program that, when executed by at least one processor, causes the at least one processor to:
control the wearable device to collect motion data of a user when executing a preset training plan;
determine an achievement rate and a completion rate corresponding to the user based on the motion data from the wearable device and the preset training plan, the achievement rate corresponding to the user characterizing an achievement situation of the user in terms of training intensity, and the completion rate corresponding to the user characterizing a completion situation of the user in terms of training volume;
determine an assessment result based on the achievement rate corresponding to the user and the completion rate corresponding to the user; and
control an electronic device to generate an output according to the assessment result.

16. The storage medium according to claim 15, wherein:
the preset training plan includes an achievement standard corresponding to a preset action; and
the at least one computer program, when executed by at least one processor, further causes the at least one processor to:
determine an achievement rate corresponding to the preset action according to the motion data and the action achievement standard corresponding to the preset action; and
determine the achievement rate corresponding to the user according to the achievement rate corresponding to the preset action.

17. The storage medium according to claim 16, wherein the at least one computer program, when executed by at least one processor, further causes the at least one processor to:
determine an achievement action meeting the achievement standard corresponding to the preset action according to the motion data and the achievement standard corresponding to the preset action; and
determine the achievement rate corresponding to the preset action according to the achievement action.

18. The storage medium according to claim 17, wherein the at least one computer program, when executed by at least one processor, further causes the at least one processor to:
obtain a standard intensity interval of the achievement standard corresponding to the preset action;
determine an intensity corresponding to the preset action according to the motion data; and
determine the achievement action according to the intensity corresponding to the preset action and the standard intensity interval.

19. The storage medium according to claim 17, wherein the at least one computer program, when executed by at least one processor, further causes the at least one processor to:
obtain a first duration corresponding to the achievement action and a second duration corresponding to the preset action, the second duration being a duration in which the intensity of the preset action specified in the preset training plan is within the standard intensity interval; and
determine the achievement rate corresponding to the preset action according to the first duration and the second duration.

20. The storage medium according to claim 15, wherein:
the preset training plan includes a completion standard corresponding to a preset action; and
the at least one computer program, when executed by at least one processor, further causes the at least one processor to:
determine a completion rate corresponding to the preset action based on the motion data and the completion standard corresponding to the preset action; and
determine the completion rate corresponding to the user based on the completion rate corresponding to the preset action.
